(19) United States
(12) Patent Application Publication
(10) Pub. No.: US 2012/0215108 A1
(43) Pub. Date: Aug. 23, 2012

(54) APPARATUS AND METHOD FOR TRACKING TUMOR FOR ULTRASOUND THERAPY AND ULTRASOUND THERAPY SYSTEM

(75) Inventors: Dong-ryeol PARK, Hwaseong-si (KR); Yeon-ho KIM, Hwaseong-si (KR)

(73) Assignee: SAMSUNG ELECTRONICS CO., LTD., Suwon-si (KR)

(21) Appl. No.: 13/244,503

(22) Filed: Sep. 25, 2011

(30) Foreign Application Priority Data
Feb. 22, 2011 (KR) 10-2011-0015572

(51) Int. Cl. A61B 8/08

(52) U.S. Cl. 600/443

(57) ABSTRACT

Apparatuses and methods for tracking a tumor for ultrasound therapy, and an ultrasound therapy system for performing ultrasound therapy are provided. The apparatus includes: a marker registration unit which registers at least one marker corresponding to a location of the tumor based on at least one location and feature of at least one lesion generated in at least one portion of the tumor designated by a therapeutic ultrasonic apparatus; a marker tracking unit which tracks the at least one registered marker from a medical image of the tumor during an ultrasound therapy and provides tracking results; and a marker renewing unit which, when at least one lesion is newly generated, renews the at least one registered marker based on at least one location and feature of the at least one newly generated lesion.

### CROSS-REFERENCE TO RELATED APPLICATION

This application claims priority from Korean Patent Application No. 10-2011-0015572, filed on Feb. 22, 2011 in the Korean Intellectual Property Office, the disclosure of which is incorporated herein in its entirety by reference.

### BACKGROUND

1. Field

The present disclosure relates to apparatuses and methods for tracking a tumor for ultrasound therapy, and an ultrasound therapy system for performing ultrasound therapy.

2. Description of the Related Art

Along with medical developments, various treatments for a tumor have been developed including invasive surgery, such as an abdominal operation, and minimally-invasive surgery. Also, currently, non-invasive surgery has been developed such as a gamma knife, a cyber knife, and a high-intensity focused ultrasound (HIFU) knife. Currently, HIFU knife is widely used as a harmless and environmental-friendly treatment since ultrasonic waves are used.

HIFU treatment using an HIFU knife is an operation performed by irradiating and focusing HIFU in a region of a tumor to cause focal destruction or necrosis of tumor tissue, thereby removing and treating the tumor. Here, in order to accurately irradiate HIFU in a region of a tumor, a medical diagnostic image accurately indicating a position of a tumor is required. Accordingly, medical images acquired from image diagnostic technology such as diagnostic ultrasonic waves, computed tomography (CT), magnetic resonance imaging (MRI), and digital angiography are used in the HIFU treatment.

### SUMMARY

One or more embodiments provide apparatuses and methods for tracking a tumor for ultrasound therapy, in particular, for high intensive focused ultrasound (HIFU) therapy, and a system for performing ultrasound therapy such as HIFU treatment. One or more embodiments also provide a computer readable recording media having embodied thereon a computer program for executing the methods.

According to an aspect of an exemplary embodiment, there is provided an apparatus for tracking a tumor for ultrasound therapy, wherein the apparatus may include: a marker registration unit which registers at least one marker corresponding to a location of the tumor based on at least one location and feature of at least one lesion generated in at least one portion of the tumor designated by a therapeutic ultrasonic apparatus; a marker tracking unit which tracks the at least one registered marker from a medical image of the tumor during an ultrasound therapy and provides tracking results; and a marker renewing unit which, when at least one lesion is newly generated, renews the at least one registered marker based on at least one location and feature of the at least one newly generated lesion.

The therapeutic ultrasonic apparatus may perform the ultrasound therapy while tracking the tumor based on tracking results provided by the marker tracking unit.

The marker renewing unit may renew the at least one registered marker by adding information about the at least one location and feature of the at least one newly generated lesion to information about existing markers registered in the marker registration unit.

The marker renewing unit may renew the at least one registered marker when at least a first part of the tumor is being treated.

The marker renewing unit may renew the at least one registered marker when at least one treated part of the tumor overlaps with the existing markers.

The marker tracking unit may track the at least one registered marker by applying a feature point tracking algorithm to the medical image.

The apparatus may further include: a treatment plan determination unit which determines a treatment plan for the tumor to be treated by the therapeutic ultrasonic apparatus based on the information about the at least one registered marker.

The treatment plan determination unit may renew the determined treatment plan based on a renewal of the at least one registered marker.

The apparatus may further include a detector which detects the at least one location and feature of the at least one lesion based on properties of the tissue where the lesion is generated, wherein the registering of the at least one registered marker is based the detecting of the detector.

The at least one registered marker may be based on an arrangement of a plurality of lesions.

According to an aspect of an exemplary embodiment, there is provided a method of tracking a tumor for ultrasound therapy, wherein the method may include: registering at least one marker that correspond to a location of the tumor based on at least one location and feature of at least one lesion generated in at least one part of the tumor designated by a therapeutic ultrasonic apparatus; tracking the at least one registered marker from a medical image of the tumor while ultrasound therapy is being conducted; and if at least lesion is newly generated, renewing the registered markers based on at least one location and feature of the at least one newly generated lesion.

The therapeutic ultrasonic apparatus may perform therapy while tracking the tumor based on the tracking of the at least one registered marker.

The renewing the at least one registered marker may include adding information about the at least one location and feature of the at least one newly generated lesion to information about at least one registered existing marker.

The renewing the at least one registered marker may include renewing the at least one registered marker when at least one part of the tumor is being treated.

The renewing the at least one registered marker may include renewing the at least one registered when at least one treated part of the tumor overlaps with the at least one registered existing marker.

The method may further include determining a treatment plan for the tumor to be treated by the therapeutic ultrasonic apparatus based on information about the at least one registered marker.

The tracking the at least one registered marker may include applying a feature point tracking algorithm to the medical image.

The method may further include detecting the at least one location and feature of the at least one lesion based on properties of the tissue where the at least one lesion is generated, wherein the at least one registered marker is based on a result of the detecting.

A computer readable recording medium having embodied thereon a computer program for executing a method which may include: registering at least one marker that correspond to a location of the tumor based on at least one location and feature of at least one lesion generated in at least one part of the tumor designated by a therapeutic ultrasonic apparatus; tracking the at least one registered marker from a medical image of the tumor while ultrasound therapy is being conducted; and if at least lesion is newly generated, renewing the registered markers based on at least one location and feature of the at least one newly generated lesion.

According to an aspect of an embodiment, there is provided an ultrasound therapy system which may include: an image acquisition apparatus which acquires a medical image of a patient; a therapeutic ultrasonic apparatus which irradiates therapeutic ultrasonic waves to at least one designated part of a tumor based on the acquired medical image, to thereby generate at least one lesion; and an apparatus which registers at least one marker corresponding to a location of the tumor based on at least one location and feature of the at least one lesion generated by the therapeutic ultrasonic apparatus, tracks the at least one registered marker from a medical image of the tumor during ultrasound therapy, and, when at least one lesion is newly generated, renewing the at least one registered marker based on at least one location and feature of the at least one newly generated lesion, wherein the therapeutic ultrasonic apparatus performs the ultrasound therapy while tracking the tumor based on the tracking of the at least one registered marker.

### BRIEF DESCRIPTION OF THE DRAWINGS

These and/or other aspects will become apparent and more readily appreciated from the following description of the embodiments, taken in conjunction with the accompanying drawings of which:

FIG. 1 is a block diagram of an ultrasound therapy system, according to an embodiment;

FIG. 2 is a block diagram of an apparatus for tracking a tumor, according to an embodiment;

FIG. 3A illustrates markers generated in a tumor according to an embodiment;

FIG. 3B is a diagram for explaining a treatment plan in consideration of markers according to an embodiment;

FIG. 4 illustrates results obtained by tracking a tumor with respect to a medical image without using markers;

FIG. 5 illustrates results obtained by tracking a tumor with respect to a medical image by using markers according to an embodiment;

FIG. 6 illustrates a method of renewing markers according to an embodiment;

FIG. 7 is a flowchart illustrating a method of tracking a tumor for ultrasound therapy according to an embodiment;

FIG. 8 is a flowchart illustrating an ultrasound therapy method according to an embodiment;

FIG. 9 is a flowchart illustrating a method of generating markers according to an embodiment; and

FIG. 10 is a flowchart illustrating a method of renewing markers according to an embodiment.

### DETAILED DESCRIPTION

Hereinafter, one or more embodiments will be described in detail with reference to the accompanying drawings.

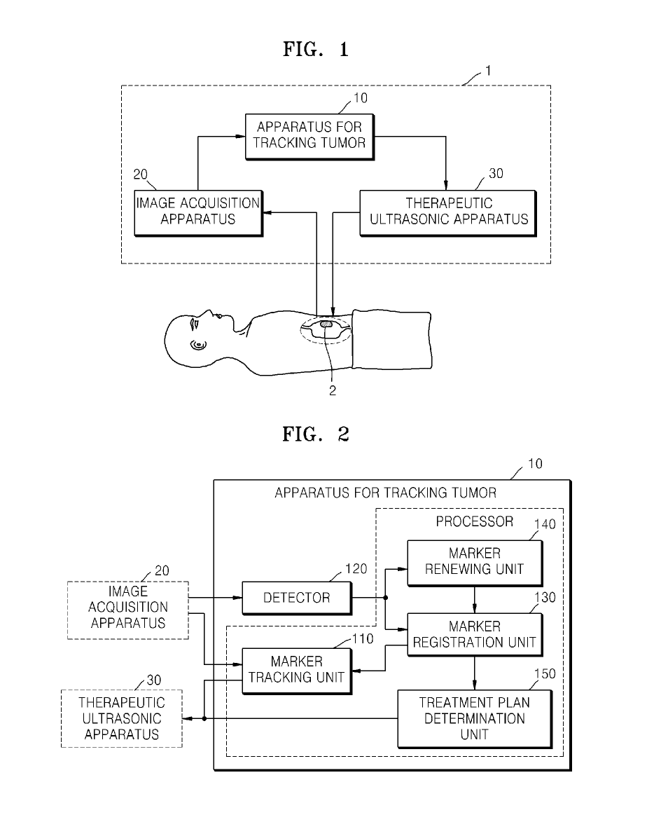

FIG. 1 is a block diagram of an ultrasound therapy system 1 for removing a tumor through ultrasound therapy, according to an embodiment. Referring to FIG. 1, the ultrasound therapy system 1 includes an apparatus 10 for tracking a tumor, an image acquisition apparatus 20, and a therapeutic ultrasonic apparatus 30.

The ultrasound therapy system 1 illustrated in FIG. 1 includes only elements related to the current embodiment. In this regard, it will be readily recognized and understood by one of ordinary skill in the art that elements other than those illustrated in FIG. 1 may be further included in the ultrasound therapy system 1.

In general, a tumor denotes cells in the body that have grown at an excessively rapid speed with autonomy. When the tumor 2 appears in the body, the tumor may spread to other tissues or internal organs and thus functions of internal organs may be damaged or paralyzed, thereby threatening peoples' health or resulting in death. In particular, the tumor may metastasize to important internal organs in the body or may become a cancer that is fatal to the body. Accordingly, it is required to treat the tumor in an early stage before the tumor spreads to other tissues or internal organs.

The ultrasound therapy system 1 according to the current embodiment identifies a location of a tumor 2 from a medical image acquired from the image acquisition apparatus 20. Then, therapeutic ultrasonic waves are irradiated from the therapeutic ultrasonic apparatus 30 to the tumor 2 and a lesion is generated by focal destruction and necrosis of tissue of the tumor 2. In particular, the ultrasound therapy system 1 according to the current embodiment performs the ultrasound therapy while the location of the tumor 2 is continuously tracked by the apparatus 10 for tracking a tumor. Elements of the ultrasound therapy system 1 will now be described.

The image acquisition apparatus 20 acquires diagnostic medical images for the diagnosis of patients. The diagnostic medical images denote medical images acquired by using image diagnostic technology such as radiation, diagnostic ultrasonic waves, computed tomography (CT), magnetic resonance imaging (MRI), digital angiography, and the like. Hereinafter, medical images acquired through diagnostic ultrasonic waves are described; however, embodiments of the present invention are not limited thereto.

The therapeutic ultrasonic apparatus 30 irradiates therapeutic ultrasonic waves to a designated part of the tumor 2 based on a medical image, thereby generating a lesion, and the patient is treated by using the therapeutic ultrasonic waves. That is, therapeutic ultrasonic waves are generated to be irradiated to local tissue of the patient.

High intensity focused ultrasound (HIFU) is used as the therapeutic ultrasonic waves according to the current embodiment. It will be readily understood by one of ordinary skill in the art that the therapeutic ultrasonic apparatus 30 according to the current embodiment generally irradiates HIFU as the therapeutic ultrasonic waves and thus a detailed description thereof will be omitted here. However, the therapeutic ultrasonic apparatus 30 according to the current embodiment is not limited to HIFU, and it will be readily recognized and understood by one of ordinary skill in the art that the therapeutic ultrasonic apparatus 30 may be any apparatus as long as the apparatus irradiates focused ultrasound similar to HIFU.

While ultrasound therapy is being conducted, the apparatus 10 for tracking a tumor continuously tracks a location of a tumor 2 in the body in real-time and thus allows continuous ultrasound therapy using the therapeutic ultrasonic waves to be conducted.

Although the patient, who is treated with the ultrasound therapy, is restricted in terms of movement, minute movement still occurs in the body, such as movement due to breathing. Accordingly, internal organs or tissue of the patient may move along with this movement and thus an accurate location of the tumor 2 may not be identified.

In this regard, in general, gating radiotherapy (RT) is used. The gating RT is performed by attaching or indicating markers on skin on the chest of the patient, thereby tracking breathing of the patient. However, such a gating RT only identifies movement of the body surface of the patient due to the breathing, and the movement of internal organs or the tumor 2 in the body may be indirectly presumed, since movement of internal organs varies according to a kind of the internal organs. Accordingly, accuracy of the therapy may be decreased.

Thus, in general, the ultrasound therapy is conducted in such a way that a patient is held by a holding bar or a patient stops breathing, that is, breathes out and holds his or her breath. However, since the therapy is able to be conducted only during the out breathing, both a doctor and a patient need to conduct the ultrasound therapy for a long period of time and thus experience inconvenience.

In addition, in general, a tool such as a gold acupuncture needle or bead is invasively inserted into organs and is captured using X-rays, thereby tracking a tumor in real-time. However, in such a method, excessive radiation may be experienced by a patient due to a continuous x-ray exposure, and various problems such as complexity of treatment and inconvenience to a patient due to invasively inserting such a tool may exist.

That is, in general, a tumor is tracked indirectly or by using an invasive method, and a tumor may not be tracked in real-time by using a non-invasive method.

In general, as a non-invasive method, a method of irradiating HIFU to the body and designating marking points has been introduced. However, such a method is used to identify a relative location for the marking points of a tumor in a patient, who is in a non-moving state, and does not track a location of a tumor in real-time due to movement of a patient.

However, the ultrasound therapy system 1 according to the current embodiment, in particular, the apparatus 10 for tracking a tumor, uses a lesion invasively generated in the tumor 2 by therapeutic ultrasonic waves (HIFU) as a marker and tracks the marker in real-time while conducting ultrasound therapy so that even if a patient moves, a location of the tumor 2 may be continuously tracked through the marker and ultrasound therapy may be performed at an accurate location of the tumor 2. Accordingly, an amount of time for performing the entire ultrasound therapy may be reduced and most of the ultrasound therapy process may be automatically performed. In addition, while the ultrasound therapy is being conducted, the apparatus 10 for tracking a tumor continuously renews markers and thus may accurately track a location of the tumor by using the renewed markers.

Hereinafter, the structure and operation of the apparatus 10 for tracking a tumor according to an embodiment will be described in detail.

FIG. 2 is a block diagram of the apparatus 10 for tracking a tumor, according to an embodiment. Referring to FIG. 2, the apparatus 10 for tracking a tumor includes a marker tracking unit 110, a detector 120, a marker registration unit 130, a marker renewing unit 140, and a treatment plan determination unit 150. The term “unit” as used herein means a hardware component and/or a software component that is executed by a hardware component such as a processor.

The marker tracking unit 110, the marker registration unit 130, the marker renewing unit 140, and the treatment plan determination unit 150 from among elements of the apparatus 10 for tracking a tumor of FIG. 2 may correspond to one or more processors. The processor may be realized by an array of logic gates or may be realized by a combination of a widely-used microprocessor and a memory, in which a program executed in the microprocessor is stored. Also, it will be understood by one of ordinary skill in the art that the processor may be realized by using other types of hardware.

The marker registration unit 130 registers a marker that corresponds to the location of a tumor based on a location and a feature of a lesion generated at the location of the tumor designated by the therapeutic ultrasonic apparatus 30. Here, the feature may include properties of tissue such as the degree of elasticity of the lesion and acoustic impedance.

The detector 120 detects the location and feature of the lesion based on the properties of the tissue where the lesion is generated. The properties of the tissue where the lesion is generated by HIFU, for example, the degree of elasticity of the lesion and acoustic impedance, are represented differently from original properties of the tissue. Such details are well known in the field of the art to which the present invention pertains and thus a detailed description thereof will be omitted here. Accordingly, the detector 120 detects the location and feature of the lesion having different properties from the original properties of the tissue by using differences in properties of the tissue.

Markers are registered in the marker registration unit 130 based on the location and feature of the lesion detected by the detector 120.

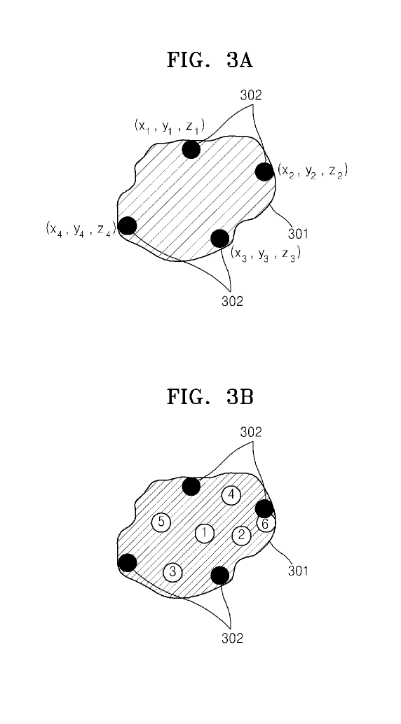

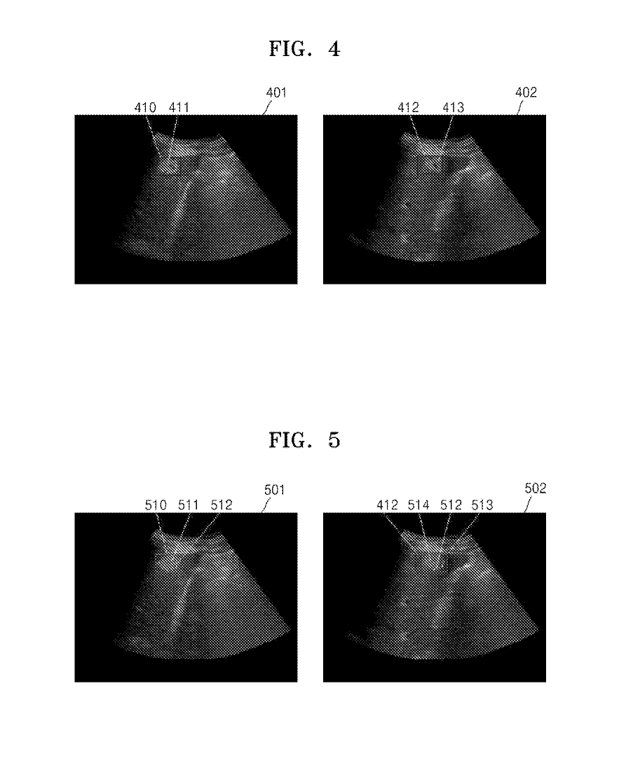

FIG. 3A illustrates markers generated in a tumor 301 according to an embodiment.

Referring to FIG. 3A, the tumor 301 includes four lesions 302. The lesions 302 are generated at designated parts of the tumor 301 by the therapeutic ultrasonic apparatus 30 of FIG. 1 and correspond to markers. A process of firstly registering markers in the tumor 301 of a patient is as follows.

First, a doctor acquires a medical image of a patient by using the image acquisition apparatus 20 of FIG. 1 and finds the location of the tumor 301 from the acquired medical image. Then, a medical examiner such as the doctor irradiates HIFU from the therapeutic ultrasonic apparatus 30 of FIG. 1 to designated parts of the tumor 301 while limiting movement of the patient, thereby generating lesions 302.

As such, the doctor generates first lesions 302 at the designated parts of the patient's tumor 301 with reference to the medical image. The detector 120 detects coordinate values of each first lesion 302 generated based on properties of the tissue where the first lesions 302 are generated, for example, spatial coordinate values of x, y, and z axes. The marker registration unit 130 registers the first markers based on spatial arrangement of the detected lesions 302.

However, as will be described with reference to FIG. 6 below, the marker registration unit 130 further registers markers renewed based on locations and features of lesions newly generated in the subsequent ultrasound therapy, in addition to the first markers illustrated in FIG. 3A.

The treatment plan determination unit 150 determines a treatment plan for a tumor to be treated using the therapeutic ultrasonic apparatus 30 based on information about the registered markers. In the ultrasound therapy using HIFU, when HIFU reaches a part of the tumor, a temperature of the part may instantaneously increase to 70° C. or more due to heat energy from HIFU. Theoretically, it is well known that tissue is destroyed at temperatures of about 70° C. or above within 110 msec. Due to such a high temperature, coagulative necrosis occurs in tissue and a blood vessel of the part of the tumor. In this case, when the high temperature of a tumor treated by HIFU does not decrease to a predetermined temperature or below, the surrounding tissue of the tumor may not be treated. Accordingly, the treatment plan determination unit 150 determines a treatment plan so that some parts of the tumor at a distance are sequentially treated by HIFU.

The treatment plan determination unit 150 according to the current embodiment may renew the determined treatment plan due to a renewal of the registered markers according to a user environment.

Reasons to use the information about the registered markers in the treatment plan are as follows. Any one of lesions indicating the registered marker has a predetermined size. However, if a part of the tumor that is overlapped with the lesion having the predetermined size is treated and thus the other overlapped lesion is generated, a location of the lesion used for the marker is confused. Accordingly, the treatment plan may be determined in consideration of the information about the registered markers.

FIG. 3B is a diagram for explaining a treatment plan in consideration of the markers 302 according to an embodiment. Referring to FIG. 3B, only a part of the treatment plan is illustrated. The treatment plan determination unit 150 determines the treatment plan for sequentially treating location \textcircled{1} to location \textcircled{6} in the tumor 301 in consideration of the locations of the markers 302. Although locations \textcircled{1} to \textcircled{6} are illustrated in FIG. 3B, the treatment plan determination unit 150 determines the treatment plan for treating the entire part of the tumor 301.

Referring back to FIG. 2, the marker tracking unit 110 tracks the registered markers from a medical image of the tumor and provides tracking results while the ultrasound therapy is being conducted. The therapeutic ultrasonic apparatus 30 receives the tracking results and treats the tumor by tracking the tumor according to the tracking results of the registered markers.

The marker tracking unit 110 tracks the markers by applying a feature point tracking algorithm to the medical image of the tumor. Here, the medical image is acquired from the image acquisition apparatus 20.

The marker tracking unit 110 tracks the markers by using a feature point tracking algorithm which uses the markers that appear in the medical image as feature points. The feature point tracking algorithm includes a tracking algorithm that is well known in the image processing field such as optical flow, speeded up robust features (SURF), scale invariant feature transform (SIFT), maximum stable extremal regions (MSER), or the like. Such an algorithm is well known to one of ordinary skill in the art to which the present invention pertains and thus a detailed description thereof will be omitted here.

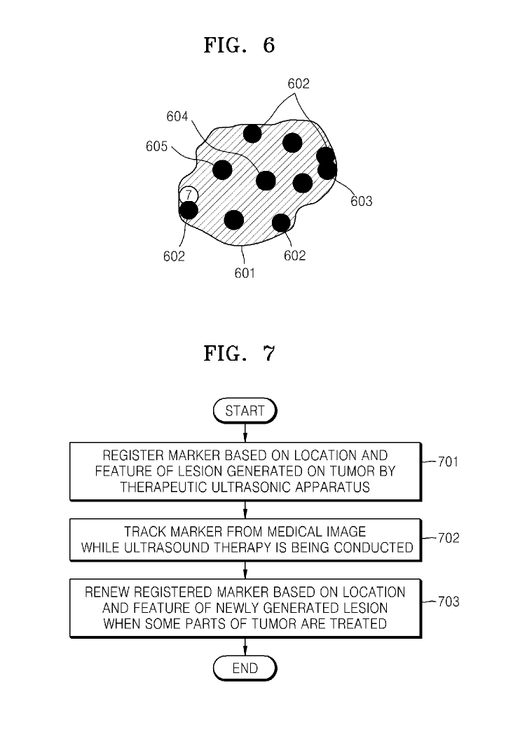

FIG. 4 illustrates results obtained by tracking a tumor 411 or 413 with respect to a medical image without using markers. Referring to FIG. 4, an ultrasonic image 401 shows when a patient stops breathing or a patient does not move so that the tumor 411 does not move, and an ultrasonic image 402 shows when a patient is breathing or a patient moves causing the tumor 413 to also move.

In the ultrasonic image 401, the tumor 411 exists in a tracking area 410. However, in the ultrasonic image 402, when markers are not used, even if the tumor 413 moves, a location of the tumor 413 may not be accurately tracked and thus the tumor 413 is not matched with a tracking area 412. Accordingly, the location of the tumor 411 or 413 may not be accurately tracked, and when the patient moves, the tumor 411 or 413 may not be accurately treated by using HIFU.

FIG. 5 illustrates results obtained by tracking a tumor 511 or 514 with respect to a medical image by using markers 512 according to an embodiment. In FIG. 5, results obtained by tracking the markers 512 by the marker tracking unit 110 are illustrated. Referring to FIG. 5, an ultrasonic image 501 shows when a patient stops breathing or a patient does not move so that the tumor 511 does not move, and an ultrasonic image 502 shows when a patient is breathing or a patient moves causing the tumor 514 to also move.

In the ultrasonic image 501, the tumor 511 exists in a tracking area 510. The markers 512 registered in the marker registration unit 130 of FIG. 2 are indicated in the tumor 511 and the marker tracking unit 110 tracks the markers 512. In the ultrasonic image 502, even if the patient is breathing or moving causing the tumor 514 to move, the markers 512 are used to accurately track the location of the tumor 514.

In comparison with the tracking area 412 in FIG. 4, in which the markers 512 are not used, the tumor 514 in the tracking area 513 in FIG. 5 is accurately tracked by using the marker 512. Accordingly, the location of the tumor 511 or 514 may be accurately tracked and even if the patient moves, the tumor 511 or 514 may be accurately treated by using HIFU.

Referring back to FIG. 2, the marker tracking unit 110 continuously tracks the markers in real-time while the ultrasound therapy is being conducted, as illustrated in FIG. 5, and provides the tracking results to the therapeutic ultrasonic apparatus 30 so that even if a tumor moves due to movement of a patient, HIFU treatment may be accurately performed on the tumor.

When some parts of a tumor are treated and thus the lesions are newly generated, the marker renewing unit 140 renews the registered markers based on locations and features of the newly generated lesions. More specifically, the marker renewing unit 140 further adds information about locations and features of newly generated lesions to existing information about the markers registered in the marker registration unit 130, thereby renewing the registered markers.

The following are three examples of the method of renewing the registered markers. According to a first example, the marker renewing unit 140 renews the registered markers when some parts of a tumor are treated. According to a second example, the marker renewing unit 140 renews the registered markers when some treated parts are overlapped with the existing markers. According to a third example, the marker renewing unit 140 renews the markers by adding newly generated lesions as a result of treating, instead of some or all lesions used in the existing markers, when some parts of a tumor are treated.

FIG. 6 illustrates a method of renewing markers 602 according to an embodiment. Referring to FIG. 6, first markers 602 are generated in a tumor 601 and lesions treated according to the treatment plan of FIG. 3B are generated in the tumor 601.

According to the first example, the marker renewing unit 140 renews the registered markers when some parts of a tumor are treated. For example, when location \textcircled{1} of the tumor 601 is treated according to the treatment plan of FIG. 3B and a new lesion 604 is generated, the marker renewing unit 140 renews the lesion 604 as a new marker along with the existing lesions 602. Then, when location \textcircled{5} of the tumor 601 is treated and a new lesion 605 is generated, the marker renewing unit 140 renews the lesion 605 as a new marker along with the existing lesions 602 and 604.

According to the first example, the marker renewing unit 140 may renew markers each time when locations \textcircled{1} to \textcircled{6} of the tumor are treated according to the treatment plan of FIG. 3B. Also, the marker renewing unit 140 may renew markers only when any specific locations of the tumor that a medical examiner, such as a doctor, desires to treat, for example, locations \textcircled{1} and \textcircled{5}, are treated. That is, a renewal of the markers according to the first example is not limited to the above description.

When the marker renewing unit 140 continuously renews new markers, in addition to the first markers, according to the first example, the tracking results of the marker tracking unit 110 may be accurate, since the markers used in tracking include more lesions and thus the number of feature points used in tracking are increased.

Then, according to the second example, the marker renewing unit 140 renews the registered markers when some treated parts overlap with the existing markers. Reasons to renew the markers according to the second example are as follows. Any one of the lesions indicating the registered markers has a predetermined size. However, if a part of the tumor that overlaps with the lesion having the predetermined size is treated and thus the other overlapped lesion is generated, a location of the lesion used for the marker may be confused.

For example, when location \textcircled{6} (referring to FIG. 3B) of the tumor 601 is treated according to the treatment plan of FIG. 3B, a newly generated lesion 603 is overlapped with the existing lesions 602. Accordingly, the marker tracking unit 110 may not accurately track since the lesions 602 and 603 are adjacent to each other. In this regard, the marker renewing unit 140 renews the existing lesions 602 and the newly generated lesion 603 as a new marker. Consequently, since the lesions 602 and 603 that are adjacent to each other are tracked once by using one marker, the location of the marker may not be confused. When the other lesion overlaps with the existing lesions 602 and is additionally generated in the subsequent therapy process, for example, when a lesion is generated in location \textcircled{7} of FIG. 6, the marker renewing unit 140 continuously renews the existing lesions 602 and the overlapped lesion as new markers.

According to the third example, the marker renewing unit 140 may renew the markers so that a lesion newly generated as a result of being treated may be used as a new marker, instead of some or all lesions used as the existing markers, when some parts of a tumor are treated. The lesions used as the existing markers may be inappropriately transformed to be used as markers in the therapy. Also, a doctor may determine the lesions used as the existing markers as inappropriate lesions to be used as markers in the therapy. In this case, the marker renewing unit 140 renews markers by replacing some or all lesions used as the existing markers with newly generated lesions.

The marker renewing unit 140 may renew markers by applying an appropriate example described above that a medical examiner, such as a doctor, desires according to a use environment.

Referring back to FIG. 2, when markers are to be renewed, the detector 120 newly detects locations and features of newly generated lesions, and the marker renewing unit 140 renews markers based on the results of the detecting. Then, the marker registration unit 130 re-registers the renewed markers as new markers to be used in tracking.

When a medical examiner, such as a doctor, desires to use only the first markers and does not desire to renew markers according to a use environment, the marker renewing unit 140 may not renew the markers.

The apparatus 10 for tracking a tumor according to the current embodiment uses a lesion invasively generated in a tumor by therapeutic ultrasonic waves (HIFU) as a marker and tracks the marker in real-time while conducting ultrasound therapy so that even if a patient moves, a location of the tumor may be continuously tracked through the marker and ultrasound therapy may be performed on an accurate location of the tumor.

FIG. 7 is a flowchart illustrating a method of tracking a tumor for ultrasound therapy according to an embodiment. Referring to FIG. 7, the method of tracking a tumor according to the current embodiment includes operations processed in time series in the apparatus 10 for tracking a tumor of FIG. 2. Accordingly, although details are omitted, details described in relation to the apparatus 10 for tracking a tumor of FIG. 2 may also be applied to the method of tracking a tumor according to the current embodiment.

In operation 701, the marker registration unit 130 registers a marker that corresponds to a location of a tumor based on a location and feature of a lesion generated at a designated part of a tumor by the therapeutic ultrasonic apparatus 30.

In operation 702, the marker tracking unit 110 tracks the registered marker from a medical image of the tumor and provides tracking results while ultrasound therapy is being conducted.

In operation 703, the marker renewing unit 140 renews the registered marker based on a location and feature of a newly generated lesion when a lesion is newly generated by treating some parts of the tumor.

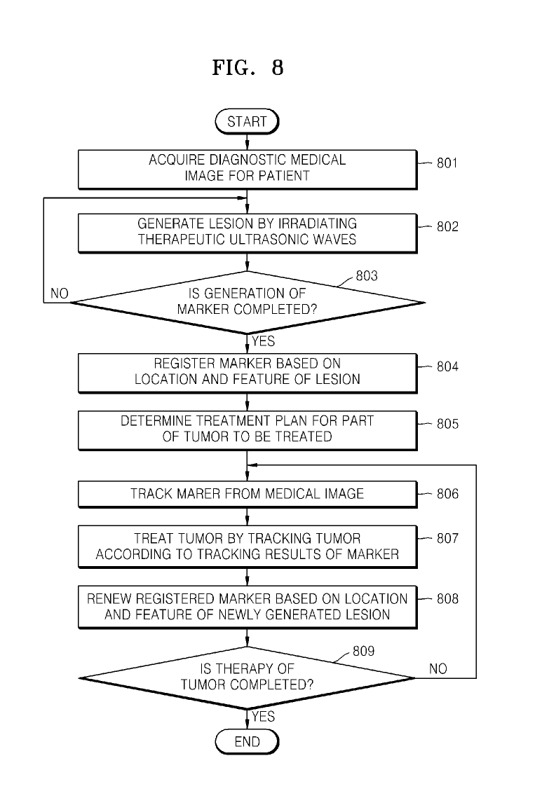

FIG. 8 is a flowchart illustrating an ultrasound therapy method according to an embodiment. Referring to FIG. 8, the ultrasound therapy method according to the current embodiment includes operations processed in time series in the ultrasound therapy system 1 of FIG. 1. Accordingly, although details are omitted, details described in relation to the ultrasound therapy system 1 of FIG. 1 may also be applied to the ultrasound therapy method according to the current embodiment.

In operation 801, the image acquisition apparatus 20 acquires a diagnostic medical image for a patient.

In operation 802, the therapeutic ultrasonic apparatus 30 irradiates therapeutic ultrasonic waves to designated parts of a tumor based on the acquired medical image and generates lesions.

In operation 803, a medical examiner, such as a doctor, identifies whether the generation of the markers is completed through the medical image. When the generation of the markers is completed, operation 804 is performed, and when the generation of the markers is not completed, operation 802 is performed again.

In operation 804, the marker registration unit 130 registers a marker that corresponds to the location of a tumor based on a location and a feature of a lesion generated at the location of the tumor designated by the therapeutic ultrasonic apparatus 30.

In operation 805, the treatment plan determination unit 150 determines a treatment plan for a part of the tumor to be treated by the therapeutic ultrasonic apparatus 30 based on information about the registered marker.

In operation 806, the marker tracking unit 110 tracks the registered maker from the medical image of the tumor and provides tracking results to the therapeutic ultrasonic apparatus 30 while ultrasound therapy is being conducted.

In operation 807, the therapeutic ultrasonic apparatus 30 treats the tumor by tracking the tumor according to the tracking results of the marker.

In operation 808, the marker renewing unit 140 renews the registered marker based on locations and features of newly generate lesions when some parts of a tumor are treated and thus the lesions are newly generated.

In operation 809, a medical examiner, such as a doctor, identifies whether therapy of the tumor is completed through the medical image. When the therapy of the tumor is completed, ultrasound therapy is completed, and when the therapy of the tumor is not completed, operation 806 is performed again.

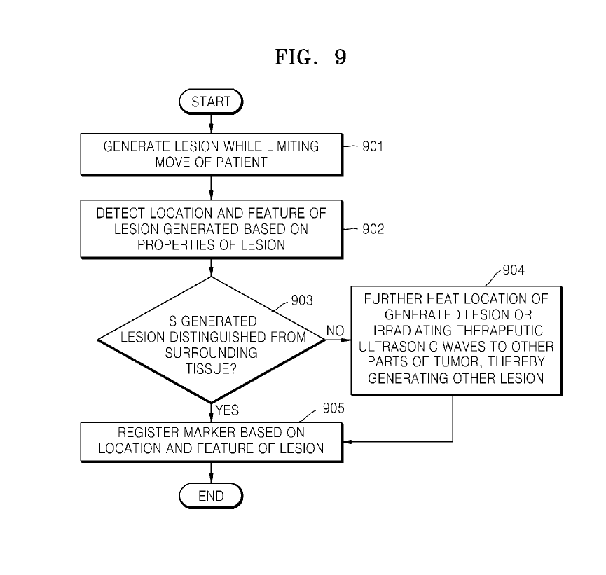

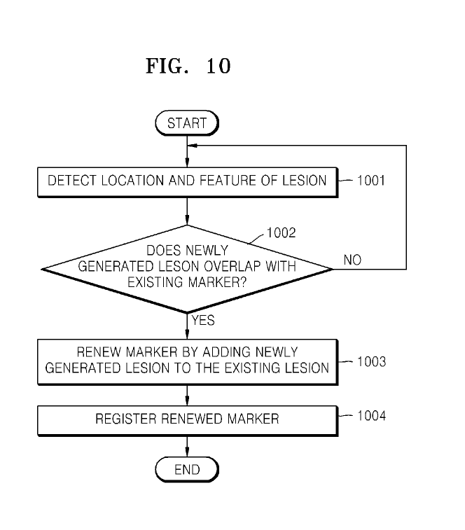

FIG. 9 is a flowchart illustrating a method of generating markers according to an embodiment.

In operation 901, the therapeutic ultrasonic apparatus 30 irradiates therapeutic ultrasonic waves to designated parts of the tumor while limiting movement of a patient, thereby generating lesions.

In operation 902, the detector 120 detects locations and features of the generated lesions based on properties of the lesions.

In operation 903, a medical examiner such as the doctor identifies whether the generated lesions may be distinguished from surrounding tissue. When the generated lesions may be distinguished from surrounding tissue, operation 905 is performed and if not, operation 904 is performed.

In operation 904, the therapeutic ultrasonic apparatus 30 further heats locations of generated lesions or irradiates therapeutic ultrasonic waves to other parts of the tumor, thereby generating other lesions.

In operation 905, the marker registration unit 130 registers a marker that corresponds to a location of the tumor based on the locations and features of the generated lesions.

FIG. 10 is a flowchart illustrating a method of renewing markers according to an embodiment.

In operation 1001, the detector 120 detects the location and feature of a lesion generated based on the properties of tissue of the lesion.

In operation 1002, the marker renewing unit 140 examines whether lesions newly generated at some treated parts overlap with existing markers. As a result, when the newly generated lesions overlap with the existing markers, operation 1003 is performed, and if not, operation 1001 is performed again.

In operation 1003, the marker renewing unit 140 renews new markers by adding newly generated lesions to the existing lesions.

In operation 1004, the marker registration unit 130 registers the renewed markers.

As described above, according to the one or more of the above embodiments, even if a patient is breathing or moving, a location of a tumor may be continuously tracked and ultrasound therapy may be performed. Also, most therapy may be automatically performed with minimum participation of a medical examiner such as a doctor and thus, time for performing the ultrasound therapy may be reduced. In addition, in order to track a tumor, non-invasive markers are used without a need for artificial insertion of separate markers to the body of a patient so that side effects and inconvenience to the patient due to insertion of invasive markers may be reduced.

The embodiments can be written as computer programs and can be implemented in general-use digital computers that execute the programs using a computer readable recording medium. Also, a data structure used in the embodiments may be recorded to a computer readable recording medium through various means. Examples of the computer readable recording medium include magnetic storage media (e.g., ROM, floppy disks, hard disks, etc.), and storage media such as optical recording media (e.g., CD-ROMs, or DVDs).

It should be understood that the exemplary embodiments described herein should be considered in a descriptive sense only and not for purposes of limitation. Descriptions of features or aspects within each embodiment should typically be considered as available for other similar features or aspects in other embodiments.

What is claimed is:

1. An apparatus for tracking a tumor for ultrasound therapy, the apparatus comprising:
a marker registration unit which registers at least one marker corresponding to a location of the tumor based on at least one location and feature of at least one lesion generated in at least one portion of the tumor designated by a therapeutic ultrasonic apparatus;
a marker tracking unit which tracks the at least one registered marker from a medical image of the tumor during an ultrasound therapy and provides tracking results; and
a marker renewing unit which, when at least one lesion is newly generated, renews the at least one registered marker based on at least one location and feature of the at least one newly generated lesion.

2. The apparatus of claim 1, wherein the therapeutic ultrasonic apparatus performs the ultrasound therapy while tracking the tumor based on tracking results provided by the marker tracking unit.

3. The apparatus of claim 1, wherein the marker renewing unit renews the at least one registered marker by adding information about the at least one location and feature of the at least one newly generated lesion to information about existing markers registered in the marker registration unit.

4. The apparatus of claim 3, wherein the marker renewing unit renews the at least one registered marker when at least a first part of the tumor is being treated.

5. The apparatus of claim 3, wherein the marker renewing unit renews the at least one registered marker when at least one treated part of the tumor overlaps with the existing markers.

6. The apparatus of claim 1, wherein the marker tracking unit tracks the at least one registered marker by applying a feature point tracking algorithm to the medical image.

7. The apparatus of claim 1, further comprising:
a treatment plan determination unit which determines a treatment plan for the tumor to be treated by the therapeutic ultrasonic apparatus based on the information about the at least one registered marker.

8. The apparatus of claim 7, wherein the treatment plan determination unit renews the determined treatment plan based on a renewal of the at least one registered marker.

9. The apparatus of claim 1, further comprising a detector which detects the at least one location and feature of the at least one lesion based on properties of the tissue where the lesion is generated, wherein the registering of the at least one registered marker is based the detecting of the detector.

10. The apparatus of claim 1, wherein the at least one registered marker is based on an arrangement of a plurality of lesions.

11. A method of tracking a tumor for ultrasound therapy, the method comprising:
registering at least one marker that correspond to a location of the tumor based on at least one location and feature of at least one lesion generated in at least one part of the tumor designated by a therapeutic ultrasonic apparatus;
tracking the at least one registered marker from a medical image of the tumor while ultrasound therapy is being conducted; and
if at least lesion is newly generated, renewing the registered markers based on at least one location and feature of the at least one newly generated lesion.

12. The method of claim 11, wherein the therapeutic ultrasonic apparatus performs therapy while tracking the tumor based on the tracking of the at least one registered marker.

13. The method of claim 11, wherein the renewing the at least one registered marker comprises adding information about the at least one location and feature of the at least one newly generated lesion to information about at least one registered existing marker.

14. The method of claim 13, wherein the renewing the at least one registered marker comprises renewing the at least one registered marker when at least one part of the tumor is being treated.

15. The method of claim 13, wherein the renewing the at least one registered marker comprises renewing the at least one registered when at least one treated part of the tumor overlaps with the at least one registered existing marker.

16. The method of claim 11, further comprising determining a treatment plan for the tumor to be treated by the therapeutic ultrasonic apparatus based on information about the at least one registered marker.

17. The method of claim 11, wherein the tracking the at least one registered marker comprises applying a feature point tracking algorithm to the medical image.

18. The method of claim 11, further comprising detecting the at least one location and feature of the at least one lesion based on properties of the tissue where the at least one lesion is generated, wherein the at least one registered marker is based on a result of the detecting.

19. A computer readable recording medium having embodied thereon a computer program for executing the method of claim 11.

20. An ultrasound therapy system comprising:
an image acquisition apparatus which acquires a medical image of a patient;
a therapeutic ultrasonic apparatus which irradiates therapeutic ultrasonic waves to at least one designated part of a tumor based on the acquired medical image, to thereby generate at least one lesion; and
an apparatus which registers at least one marker corresponding to a location of the tumor based on at least one location and feature of the at least one lesion generated by the therapeutic ultrasonic apparatus, tracks the at least one registered marker from a medical image of the tumor during ultrasound therapy, and, when at least one lesion is newly generated, renewing the at least one registered marker based on at least one location and feature of the at least one newly generated lesion,
wherein the therapeutic ultrasonic apparatus performs the ultrasound therapy while tracking the tumor based on the tracking of the at least one registered marker.
